(19) United States
(12) Patent Application Publication
(10) Pub. No.: US 2026/0162389 A1
(43) Pub. Date: Jun. 11, 2026

(54) METHOD FOR REGISTRATION OF A VIRTUAL IMAGE IN AN AUGMENTED REALITY SYSTEM

(71) Applicant: Brainlab SE, Munich (DE)

(72) Inventors: Hagen Kaiser, Munich (DE); Stefan Vilsmeier, Munich (DE)

(21) Appl. No.: 18/842,625

(22) PCT Filed: Mar. 25, 2022
(86) PCT No.: PCT/EP2022/057975

§ 371 Date: Aug. 29, 2024

(51) Int. Cl. G06T 19/20; A61B 34/20; G06T 7/30

(52) U.S. Cl. CPC G06T 19/20; G06T 7/30; A61B 34/20

(57) ABSTRACT

Disclosed is a method for registration of a virtual image in an augmented reality, AR, system comprising an AR device. The method comprises acquiring a first image by means of a camera of the AR system, wherein the first image depicts a first projection of a first laser beam onto a surface of a subject, the first projection being in the shape of a first line representing the intersection of a first plane with the surface of the subject, the first plane arranged such that it comprises or has a fixed spatial relation with respect to a target point; determining, based at least on a shape and/or position of the first line in the first image, a first component of a position of the target point in AR device coordinates; and adjusting a registration of a virtual image, which is to be overlaid with the field of view of the AR device, based on the first component of the position of the target point in AR device coordinates.

### FIELD OF THE INVENTION

The present invention relates to a method for registration of a virtual image in an augmented reality, AR, system comprising an AR device, an AR system, a medical system comprising the AR system, a computer program, a program storage medium, and a computer, a signal wave, and a data stream.

### TECHNICAL BACKGROUND

Augmented reality is a technology that superimposes information relative to the reality that the user of an augmented reality device is situated in. To allow for reliably superimposing the information, the augmented reality device has to localize itself relative to its environment.

There are certain environments that allow for particularly reliable localization and registration. For example, when applying augmented reality in a room, the walls, the floor, and/or furniture, for example, can be used in combination with image data from one or more cameras and/or 3D scanners, optionally complemented by acceleration data and/or gyroscopic data of the augmented reality device. This allows for a relatively reliable and stable localization and registration. As an example, the augmented reality device may perform localization using an inbuilt camera system to scan the room and utilizing computer vision methods.

The above methods are usually sufficiently precise, for example, for gaming purposes or the like. However, the precision may not be sufficient in certain professional applications that require particularly precise registration of virtual images, for example in cases where augmented reality is used for high precision interaction of a user of the augmented reality device with their environment, e.g., in the medical field.

There are certain cases, where the method of using features of the room, like the walls, the floor, and/or the furniture, may not be sufficient to achieve this precision. Moreover, the environment wherein the augmented reality system is used may be dictated by constraints brought about by the given application of the augmented reality system. For example, features like walls, the floor, and/or furniture may not always be within the field of view (FOV) of the augmented reality device or may not or only partially be visible due to machine components and instrumentation required for the given application that may block the view.

Some proposed methods have made use of markers placed in advance of using the augmented reality system, which may then be used as a reference point for localization. However, depending on the application of the augmented reality system, the placement of the markers may be difficult. As an example, there may be applications where the field of view changes, such that any previously positioned markers may not remain in the field of view. Moreover, when objects in the real environment are moved around, this may lead to markers being covered up or even being moved from their original position. In these cases, localization may not be reliably ensured at the required precision.

As can be seen from the above, there may be certain cases where currently known methods fail to provide precise and stable localization and registration, thereby limiting the fields of application for the augmented reality system.

It is an object of the present invention to provide a method for image registration in an augmented reality system that allows for reliable use of the augmented reality system over an extended range of potential applications. It is also an object of the present invention to provide an augmented reality device and system configured so as to be reliably used over an extended range of potential applications.

The present invention can be used for essentially all applications, even those that require precise localization to reliably support a user's interaction with their environment. Such applications may include preparing for selectively exposing a subject to radiation, which requires high precision in terms of positioning the subject and the radiation source in such a manner that the radiation exposes precisely the correct portion of the subject. Such procedures may be performed, for example, in connection with a system for image-guided radiotherapy such as VEROR and ExacTrac®, both products of Brainlab AG. However, the procedures may also be used for other types of positioning, for example of machine components or instrumentation, particularly in professional applications, including, for example, industrial applications.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The present invention provides a method for registration of a virtual image in an augmented reality, AR, system comprising an AR device, an AR system, a medical system comprising the AR system, a computer program, a program storage medium, and a computer, a signal wave, and a data stream according to the independent claims. Preferred embodiments are laid down in the dependent claims.

The method for registration of a virtual image in an augmented reality, AR, system comprising an AR device comprises acquiring a first image by means of a camera of the AR system, wherein the first image depicts a first projection of a first laser beam onto a surface of a subject, the first projection being in the shape of a first line representing the intersection of a first plane with the surface of the subject, the first plane arranged such that it comprises or has a fixed spatial relation with respect to a target point. The method further comprises determining, based at least on a shape and/or position of the first line in the first image, a first component of a position of the target point in AR device coordinates. The method further comprises adjusting a registration of a virtual image, which is to be overlaid with the field of view of the AR device, based on the first component of the position of the target point in AR device coordinates.

### GENERAL DESCRIPTION OF THE INVENTION

In this section, a description of the general features of the present invention is given for example by referring to possible embodiments of the invention.

The present disclosure provides a method for registration of a virtual image in an augmented reality, AR, system comprising an AR device. The method comprises acquiring a first image by means of a camera of the AR system, wherein the first image depicts a first projection of a first laser beam onto a surface of a subject, the first projection being in the shape of a first line representing the intersection of a first plane with the surface of the subject, the first plane arranged such that it comprises or has a fixed spatial relation with respect to a target point. The method further comprises determining, based at least on a shape and/or position of the first line in the first image, a first component of a position of the target point in AR device coordinates. The method further comprises adjusting a registration of a virtual image, which is to be overlaid with the field of view of the AR device, based on the first component of the position of the target point in AR device coordinates.

The present invention leverages the knowledge of a relative spatial arrangement of the target point and a plane defined by the first laser beam may be used together with the knowledge that a projection of a laser beam onto the surface of a subject will have certain properties, e.g., be shifted and/or distorted depending on the shape of the subject's surface. Based thereon and the known relative arrangement of the target point and the plane, it can be determined whether a current first component, e.g., an x-, y-, or z-coordinate, of the target point in AR device coordinates corresponds to that derived from the first image or whether the coordinate requires adjustment.

It is to be understood and will be seen from the discussion below that this can be similarly applied for the other components of the position of the target point. Moreover, it is to be understood that this may be supplemented by other data, for example, a position of a crossing of projections on the subject's surface obtained from the first image or one or more additional images, or data obtained by other sensors, as will be described in more detail further below. E.g., known methods of localization, for example employing gyroscopic methods and/or inside out tracking methods of augmented reality systems may be used in addition to the localization based on the projection(s). Thus, there may be sufficient data to perform localization for all directions even if not all of the projections are currently visible. The projection(s) that is/are visible will improve any result obtained from said known methods. Thereby, reliability and precision will already be improved over localization and registration performed relying only on said known methods.

An advantage of using the laser projection in the manner disclosed herein for the registration is that precise registration and, consequently, localization of the augmented reality device, can be achieved in a wide variety of scenarios.

For example, the registration is applicable even for very limited and/or changing fields of view that do not reliably comprise walls or other fixed reference points, or when the augmented reality device or the subject or other objects are moved such that potential reference points may not always be visible. As an example, when the shape of the laser projection is used for the registration, even when part of the room or the subject that might otherwise serve as a reference point for registration is not in the field of view or is obscured, the laser projection may still be evaluated either by looking at the non-obscured portions thereof or by taking into account the shape of the obscuring object.

In the present disclosure, any relative or absolute arrangements refer to the arrangement for designated use unless otherwise specified. Acquiring images, in the present disclosure and unless explicitly specified otherwise, refers to the recording of the image data. That is, when an image is acquired at a point in time, it represents the depicted scene at said point in time.

As can be seen from the above, the method of the present disclosure allows for reliable use of the augmented reality system over an extended range of potential applications including high-precision applications.

The AR system of the present disclosure comprises an AR device and a camera. The camera may be part of the AR device or connected to the AR device. The AR device may comprise a see-through display and virtual images may be overlaid on the reality as viewed by a user through the see-through display. For example, the AR device may be configured as smart glasses. Alternatively or in addition, the AR device may be configured to display a live image and overlay the live image with a virtual image. In this case, the display may or may not be see-through. In each of the cases, an exact registration to match the virtual image with the reality as viewed by a user through the see-through display and/or the live image is required for the augmented reality system to improve usefulness for high precision applications.

The camera may be configured to acquired 2D image data. The first image may be a 2D image. Similarly, the second image described further below may be a 2D image, e.g., obtained by the/a camera configured to acquire 2D image data.

The target point of the present disclosure may be used as a reference for transformations of images and/or coordinate systems that are performed as part of and/or prior to the registration. The target point may, for example, be an isocenter of the room in which the system is installed or may be a machine isocenter, but may be any other suitable point as well. As an example, a point that is determined to likely remain in the field of view of the camera—albeit obscured by the subject—may be selected as a target point.

The first laser beam may be emitted by a first laser that provides a fan laser geometry. For example, the laser beam may be diverged or scanned along a line, such that the laser beam defines a plane and, when projected onto a flat surface, the projection has the shape of a straight line.

According to the present disclosure, the first plane is arranged such that it comprises or has a fixed spatial relation with respect to a target point. This may be achieved by defining the target point first and then setting up and calibrating the first laser providing the first laser beam so as to obtain the spatial relation, for example in such a manner that the first plane comprises the target point. Alternatively, a target point may be defined only after setting up and calibrating the first laser.

In particular, there may be a first laser beam defining the first plane and a second laser beam defining a second plane and the target point may be located on the line where the first and second plane intersect. More particularly, a third laser beam defining a third plane may be provided and the point where all three planes intersect may be the target point. Each of the lasers of the present disclosure may be a room laser, e.g. as found in the medical field, e.g., in operating rooms.

In case there are three planes as described above, optionally the three planes may be orthogonal planes. They may thus span a coordinate system, e.g., with the target point as an origin.

The registration of the virtual image in an augmented reality system may be achieved, as an example, by transforming coordinate systems, preferably using the target point as a reference point, for example, as a common origin.

Adjusting a registration, according to the present disclosure, is performed based on the first component of the position of the target point in AR coordinates. For example, this may entail shifting the virtual image so as to have a predetermined position with respect to the target point in AR device coordinates, specifically changing the first component of the virtual image's position by an amount derived from the first component of the target point in AR coordinates. As an example, the origin of the virtual image may be shifted. When the registration is adjusted based on two or more of the components of the position of the target point, the virtual image may additionally be shifted in other directions, in particular, such that its origin matches the target point in AR device coordinates. Additional transformations, e.g., rotations and/or scaling of the virtual image may be additionally be performed.

Optionally, equipment may be aligned using the target point as a reference for the alignment as well. In particular, a piece of equipment may be configured and arranged such that the isocenter of the piece of equipment corresponds to the target point.

As mentioned above, the projection of the first laser beam onto the surface of the subject is depicted in the first image. This may be achieved by suitable arrangement of the subject and the camera relative to the first laser beam. For example, the subject may be arranged such that the laser beam is projected onto the surface of the subject and the camera may be arranged such that its field of view depicts at least part of, preferably all of the projection.

The projection will be depicted as a line in the image. The shape and/or position of the line in the image may depend, among others, on the shape of the surface of the subject, and/or the arrangement of the camera and/or the viewing angle of the camera, as well as the arrangement of the first plane.

The method may leverage available information on the shape and/or position of the subject, and/or arrangement of equipment and devices, e.g., the camera, for improving efficiency and/or accuracy of deriving components of the target point in AR coordinates based on the shape and/or position of the first line. For example, ambiguities may be resolved or potential candidates may be ruled out. There are various possible ways of doing so, which will be discussed in more detail further below.

It is noted that the method of the present disclosure is particularly advantageous in the context of applications in an environment where room lasers are already present, for example for calibration of machine positioning, as the method can then be seamlessly integrated into the existing systems without requiring, for example, additional hardware.

The method of the present disclosure is particularly advantageous for environments in which machines are calibrated with respect to an isocenter, for example the isocenter of a machine that emits a radiation beam. In these types of environments, machine components and the subject or object to be exposed to the radiation beam may be generally positioned relative to the isocenter. In particular, the parts of the subject or object to be radiated has to be placed into the isocenter where the radiation beam is located. This also means, that any physical placement of markers in the isocenter could be detrimental to the application at hand. In this type of application, usually a large part of the field of his covered by movable elements, such as the subject or object itself and movable machine parts in case of a radiation machine, for example the gantry of the linear accelerator is a movable part that is relatively large and located near the isocenter. Accordingly, this type of environment is particularly challenging for reliably and precisely localizing the augmented reality device with respect to its real environment and registering the virtual image. The room lasers provide beams that have a well-defined orientation within the room, such that the projections will remain visible even when large parts of the environments move around, are well-suited for reliable localization and registration.

For the sake of completeness, it is noted that the method of the present disclosure may be a computer-implemented method.

According to the present disclosure, the first image may further depict a second projection of a second laser beam onto the surface of the subject, the second projection being in the shape of a second line representing the intersection of a second plane with the surface of the subject. An intersection of the first plane and the second plane may comprise the target point or have a fixed spatial relation with respect to the target point. The method of the present disclosure may comprise determining, based at least on a shape and/or position of the second line in the first image, and optionally also based on the shape and/or position of the first line in the first image, a second component of the position of the target point in AR device coordinates. The adjusting of the registration of the virtual image may be performed based on the first component and the second component of the target point in AR device coordinates.

As an example, if a second component of the position of the target point in AR device coordinates is to be adjusted, using only the first projection may not yield the desired accuracy. Using a second laser beam in the manner of the present disclosure allows for a more accurate adjustment of the second component. The second projection may be used essentially in the same manner for adjusting the registration on the basis of the second component as the first projection is used for adjusting the registration on the basis of the first component, e.g., by shifting an image and/or coordinate system.

Optionally, the first projection may be used in addition to the second projection for adjusting the second component. For example, the relative arrangement and/or a crossing of the first line and the second line may be used and/or the shape and/or position of the first line may be used in addition to the shape and/or position of the second line. Thus, for example, compared to using only the shape and/or position of the second line, accuracy of determining the second component may be increased and/or simplified by providing more information and constraints. Similarly, the second line may also be used in adjusting the first component, e.g., based on a known relation of the planes. This may improve overall accuracy.

The first plane and second plane may be perpendicular and, in particular, the target point may be comprised in their intersection.

The features and considerations outlined above with respect to the first laser beam, the first laser, the first plane, the first line, and the first component essentially apply accordingly to the second laser beam, the second laser, the second plane, the second line, and the second component.

The method of the present disclosure may further comprise determining a third component of the position of the target point in the AR device coordinates and the adjusting the registration of the virtual image may be performed based on the first component, the second component, and the third component of the position of the target point in AR device coordinates.

Thus, all three components may be adjusted, i.e., a complete adjustment of in all three dimensions is possible. This may be done in a similar manner as outlined in the context of the first and second component.

The method may further comprise obtaining an image that depicts a third projection of a third laser beam onto the surface of the subject, wherein the third projection is in the shape of a third line representing the intersection of a third plane with the surface of the subject, the third plane being arranged such that the intersection of the first plane, the third plane, and optionally the second plane, comprises the target point or has a fixed spatial relation with respect to the target point. The method may further comprise determining, based at least on a shape and/or position of the third line in the image, and optionally also based on the shape and/or position of the first line and/or the second line, the third component of the position of the target point in AR device coordinates.

Obtaining the image may comprise accessing an image, for example the first image, or acquiring an additional image, for example, the second image.

Thus, similarly to the first line and/or the second line, the third line can be used alone or in combination with information pertaining to the first and/or second line, to adjust one of the components, in this case, the third component, e.g., an intersection and/or relative orientation. The features and considerations outlined above with respect to the first and/or second laser beam, the laser, the plane, the line, and component essentially apply accordingly to the third laser beam, the third laser, the third plane, the third line, and the third component.

The image may be the first image or a second image acquired by means of the camera of the AR system and depicting at least one of the first projection and the second projection. Particularly, the second image may be an image acquired at the same time as the first image

Accordingly, the image that depicts the third projection and is used for adjusting the third component may be the first image that also depicts the first and optionally the second projection or it may be a different second image that, in addition to the third projection, depicts the first and/or second projection. The latter helps providing additional spatial information.

Allowing for the third component to be adjusted on the basis of a second, different image may be advantageous because, depending on the arrangement of the camera and its field of view, not all projections may be visible within the same image or only a small portion may be visible or their depiction in the image may not be ideal for deriving the third component.

In other words, the method may comprise using at least two images, particularly each depicting a subset of the projections, in particular an overlapping subset, the subset selected, for example, so as to allow for establishing a spatial relation between the images. The latter may not be necessary in case such a spatial relation can be established in some other manner, e.g., if the two images have a known relative spatial arrangement.

According to the present disclosure, the determining of the first component and/or the determining of the second component and/or the determining of the third component of the position of the target point in AR device coordinates may further be based on a shape of the surface of the subject obtained from 3D image data of the surface.

As already explained above, the shape and/or position of the projection, more specifically of the line, depends on the shape of the surface onto which the laser beam is projected. For example, a surface that is entirely flat will yield a straight-line projection. On the other hand, a curved surface will yield a projection that does not appear as a straight line in an image unless the viewing direction of the camera is parallel to the plane of the laser beam. Accordingly, generally the shape of the line in the image and an expected shape of the surface can be used to reconstruct the plane intersecting the surface.

It is possible to model the expected shape of the surface. Improved accuracy may be possible when using actual 3D image data of the surface. The 3D image data may be live 3D image data, i.e., acquired at the time of acquiring the first image and/or second image. This allows for relatively direct mapping of data.

Alternatively, the 3D image data may be data obtained prior to the first and/or second image data. This allows for performing a certain amount of image processing in advance and for more flexibility in terms of imaging modalities.

The 3D image data of the surface may comprise image data obtained by a surface camera (e.g., a depth camera) and/or image data obtained by a surface scanner, and/or contour data obtained from CT or MRI images.

As an example, 3D image data of the surface may be registered with the first and/or second image. Thus, the 3D image data may be used to add depth information to the first and/or second image, such that the 3D shape of the projections and/or the orientation of the respective plane can be directly derived from the respective line in the first and/or second image and the corresponding depth information.

According to the present disclosure, the determining of the first component and/or the second component and/or the third component of the position of the target point in AR device coordinates may comprise determining, based on the shape of the surface of the subject, a plurality of candidate lines and/or a plurality candidate crossing positions of crossings of candidate lines in the first image and/or in the second image.

The determining of the first component and/or the second component and/or the third component of the position of the target point in AR device coordinates may further comprise matching the first line and/or the second line and/or the third line and/or one or more crossing positions of crossings of the first line and/or the second line and/or the third line in the first image and/or in the second image with the plurality of (the) candidate lines and/or the plurality of candidate crossing positions, so as to obtain one or more matching candidate lines and/or one or more matching candidate crossing positions.

The determining of the first component and/or the second component and/or the third component of the position of the target point in AR device coordinates may further comprise determining the first component and/or the second component and/or the third component of the position of the target point in AR device coordinates based on the one or more matching candidate lines and/or the one or more matching candidate crossing positions.

A candidate line may be representative of the intersection of a corresponding candidate plane with the surface. A candidate crossing position may be the position of a crossing of two or more candidate lines, and, accordingly, marks the intersection of two or more candidate planes.

Based on the matched candidate lines and/or crossing positions and using the fixed spatial relation of the target point and the first plane and/or the second plane and/or the third plane and/or their intersections, one or more of the components of the position of the target point in AR device coordinates can be determined. Optionally, candidates may be eliminated based on a known relative arrangement of the planes.

As one example, described in more detail below, one or more candidate planes corresponding to the matched candidate line(s) and or candidate crossing position(s) may be used to reconstruct the first plane and/or second plane and/or third plane. One or more of the components of the position of the target point in AR device coordinates can be determined using the fixed spatial relations and the reconstructed first plane and/or second plane and/or third plane.

The matching of the first line and/or second line and/or third line with the candidate lines may be based on the shape and/or position of the respective line.

As mentioned above, shape and/or position of a projection the laser beam in the first and/or second image depend on the shape of the surface onto which the laser beam is projected. Accordingly, predictions for the shape of the projections can be made for multiple candidate planes. The predictions may be used for matching lines depicted in the first image and/or the second image with candidate lines and, optionally, selection of corresponding candidate planes. Relative arrangement of a given candidate line and/or a corresponding candidate plane relative to the target point may be known and based thereon, the respective component of the position of the target point in AR device coordinates can be determined.

Alternatively or in addition, a candidate crossing of the projections of two candidate planes and its position can be predicted for multiple pairs of candidate planes. This may be used for matching crossing positions detected in the first image and/or second image with candidate crossing positions. The positions of the crossings may thus be obtained in AR device coordinates and based on information where the crossings are arranged relative to the target point, one or more components of the target point position may be derived.

The method of the present disclosure may comprise reconstructing the first plane from the shape of the first line in the first image, wherein determining the first component of the position of the target point in AR device coordinates is based at least on the reconstructed first plane. Alternatively or in addition, the method of the present disclosure may comprise reconstructing the second plane from the shape of the second line in the first image, wherein determining the second component of the position of the target point in AR device coordinates is based at least on the reconstructed second plane. Alternatively or in addition, the method of the present disclosure may comprise reconstructing the third plane from the shape of the third line in the image, in particular the first image or the second image, wherein determining the third component of the position of the target point in AR device coordinates is based at least on the reconstructed third plane.

The reconstructed planes are planes in AR device coordinates. Based on the reconstructed plane(s) and the fixed relation of the plane(s) or the intersection(s) and the target point, the respective component of the position of the target point in AR device coordinates can be determined.

For example, multiple lines may be depicted in an image, and a known fixed relation between the planes, e.g., that the planes are orthogonal planes, may be used as supplementary information. For example, when the image depicts one or more crossings, the potential set of candidate planes is limited when knowing that they must have a fixed relation.

Reconstructing the planes may, for example, be performed by matching candidate lines and/or candidate crossing positions as described above and identifying the candidate planes corresponding to the matched candidate lines and/or candidate crossing positions as the first and/or second and/or third plane and/or using live 3D image data to add spatial information.

According to the present disclosure, the reconstructing of the first plane and/or the second plane and/or the third plane may further be based on a/the shape of the surface of the subject obtained from 3D image data of the surface, and/or a position of the first line and/or the second line and/or the third line in the first image and/or the second image, and/or a relative position of the first line with respect to the second line and/or the third line in the first image, and/or a relative position of the first line and/or the second line with respect to the third line in the second image, and/or one or more crossing positions of the first line and/or the second line and/or the third line in the first image and/or in the second image.

The 3D image data of the surface may be any of the 3D image data described above. As an example, the 3D image data of the surface may be used for reconstructing the planes by matching candidate lines and/or candidate crossing positions as described above and identifying the candidate planes corresponding to the matched candidate lines and/or candidate crossing positions as the first and/or second and/or third plane.

Alternatively or in addition, the 3D image data of the surface may be registered with the first and/or second image. Thus, the 3D image data may be used to add depth information to the first and/or second image, such that the orientation of the respective plane can be directly derived from the respective line and the corresponding depth information.

According to the present disclosure, the determining of the first component and/or second component and/or third component of the position of the target point in AR device coordinates may further be based on additional measurement data, in particular data from the camera and/or from an additional camera, particularly a surface camera, and/or image data from other imaging means, particularly CT or MRI imaging means, and/or gyroscopic tracking data obtained by the augmented reality system and/or inside-out tracking data.

For example, live 3D image data may be used, i.e., image data depicting the subject at the time of acquiring the first image and optionally the second image. As explained above, 3D image data of the surface may be used in determining the position of the target point, e.g., by adding 3D information to the 2D first and optionally 2D second image and/or for determining target lines and/or planes. The 3D image data may be obtained using any of the above-mentioned imaging means. Alternatively or in addition image data may be used for establishing the arrangement and/or spatial relations of the subject, pieces of equipment, and/or the augmented reality device. This may improve overall accuracy of the determination. Similarly, using data that tracks the position and/or movement of the AR device may improve overall accuracy of the determination.

Alternatively or in addition, according to the present disclosure, the determining of the first component and/or second component and/or third component of the position of the target point in AR device coordinates may further be based on information on the shape of the subject and/or arrangement of the subject relative to the camera and/or the target point. This may improve accuracy by providing additional spatial information, as explained above.

Alternatively or in addition, according to the present disclosure, the determining of the first component and/or second component and/or third component of the position of the target point in AR device coordinates may further be based on position information on a marker located on the subject and depicted in the first image and/or second image.

In particular, one or more markers may be located on the subject to identify reference points on the surface of the subject that may be used particularly when the shape of the surface of the patient is used for determining the position of the target point. Accordingly, it may not be necessary to acquire live 3D image data of the surface of the subject for performing the above-described steps that leverage the shape of the surface. Instead, the candidate lines and candidate crossing positions may be determined from pre-recorded 3D image data that also depict the marker. The marker may make it easier to use these candidate lines and crossing positions determined from the pre-recorded 3D image data at a later time, e.g. a time of localization and registration. Alternatively or in addition, the marker may make it easier to register live or pre-recorded 3D image data with the first and/or the second image for adding depth information. Moreover, depending on the marker, distances and/or viewing angles of a/the camera with respect to the marker may be determined as supplementary data for the registration.

The present disclosure may leverage the fact that in many cases the arrangement of the subject with respect to the target point is planned in advance and optionally the shape of at least part of surface of the subject may have been determined in advance. While the subject may, at the time of adjusting the registration, not be exactly in the planned position and the shape of the surface may have slightly changed, the information may still be used for narrowing down potential candidate lines and/or planes to increase efficiency and/or resolve ambiguities and/or perform a plausibility check for steps leading up to or comprised in the adjusting of the registration.

According to the present disclosure, the determining of the first component and/or second component and/or third component of the position of the target point in AR device coordinates may further be based on information on an arrangement of the first plane and/or the second plane and/or the third plane, in particular an arrangement relative to each other and/or the subject and/or the camera and/or the target point. This may serve to increase efficiency and/or resolve ambiguities and/or increase accuracy. As an example, the planes may be orthogonal planes. Alternatively or in addition, one or more of the planes may be arranged vertically or horizontally. The planes may intersect at the target point. Reference is made to the above examples as well.

Alternatively or in addition, according to the present disclosure, the determining of the first component and/or second component and/or third component of the position of the target point in AR device coordinates may further be based on previously determined values of the first component and/or second component and/or third component of the position of the target point in AR device coordinates and/or information on at least one of a previously determined reconstructed first plane and/or second plane and/or third plane.

This may be useful when a subject is monitored and it is to be expected that the subject's position and/or the position of the AR device will change in a predictable manner and/or within a predictable range in a given period of time. In that case, many determinations made for determining the target point at a first point in time may be re-used for subsequent points in time. For example, when three intersecting lines are shown in the first image at a first point in time and these three intersecting lines have been identified with a corresponding plane, even if the lines have a slightly different shape and/or position in the next image, the general correlation of the lines with the planes will still likely be correct. Thus several calculation steps may be omitted by making use of previously determined information and/or ambiguities may be resolved.

Alternatively or in addition, according to the present disclosure, the determining of the first component and/or second component and/or third component of the position of the target point in AR device coordinates may further be based on information obtained by means of a calibration procedure.

For example, information may by obtained by means of a calibration of the AR device and/or calibration of one or more of the lasers and/or calibration of one or more other pieces of equipment, particularly cameras, surface scanners, and/or other imaging means. As calibration procedures are routinely carried out for many pieces of equipment and often leverage and/or yield spatial information, using information obtained by means of a calibration procedure may enhance the speed and/or accuracy of determining the position of the target point with little or no additional effort. Reference is particularly made to the features and advantages outlined in the context using previously determined values, which also apply in this context.

The method of the present disclosure may comprise continuously or at predetermined times and/or time intervals repeating the steps of acquiring/obtaining the first image, and optionally the second image, determining at least one of the first component, the second component, and the third component of the position of the target point in AR device coordinates, determining whether at least one of the first component, second component, and third component of the position of the target point in AR device coordinates differs from a previously determined and stored first component, second component, and third component, respectively, of the position of the target point in AR device coordinates, and, in response to determining that this is the case, updating at least one of the previously determined and stored first component, second component, and third component.

In other words, the localization and registration can be monitored over time and adjusted if they have deteriorated. In a step of determining the position of the target point in AR device coordinates, one or more previous positions of the target point and/or other data obtained in the process of and/or in preparation for determining the previous position of the target point may be used, particularly, may be used to replace one or more data processing steps that would otherwise be required for determining the position of the target point. Reference is particularly made to the features and advantages outlined in the context using previously determined values, which also apply in this context. When performing monitoring, in order to avoid delays, it is particularly advantageous to leverage previously determined data, as the overall computational load is reduced and/or speed increased.

The method of the present disclosure may comprise calculating, based on measurements performed in advance of placing the subject and based on the target position and orientation of the subject with respect to the target point or based on measurements determining the surface of the subject and placement of the subject relative to the laser beams when the subject is placed, the expected projections of each of the laser beams.

For example, a target position and orientation of the subject with respect to the first, second, and third plane and/or with respect to the target point, and the shape of the surface of the subject where the first, second, and third plane will intersect the subject if paced in the target position may be used to predict the expected projections of the laser beams, in particular, to predict the expected shape of the first, second and/or third line.

Similar steps as outlined above for obtaining the candidate lines may be applied for predicting the expected shape of the respective line.

This means, among others, that if the subject is correctly aligned in the target position and the AR device is correctly localized and the virtual image correctly registered, the lines as depicted in the first and/or second image will match the expected projections. Thus, alignment and/or registration may be verified.

The method of the present disclosure may further comprise that the AR device displays, as part of the virtual image, the expected projections of the laser beams, so as to allow for a user of the AR device to determine whether the expected projections and the actual projections match. For example, it may be immediately apparent whether or not the registration and subject position are in order. Assuming the subject position is in order, it will be immediately apparent that registration requires correction when there is no match. Assuming the registration is in order, it will be immediately apparent that the subject is misaligned, and vice versa. Thus, this feature allows for recognizing if verifying registration and/or subject alignment may be necessary.

Alternatively or in addition, the method of the present disclosure may further comprise determining, from an image obtained by means of the camera, that the projections of each of the laser beams deviate from the expected projection, and, in case that the deviation exceeds a predetermined threshold, outputting an alert and/or taking automatic correction steps.

For example, image data from a camera or other imaging means with a fixed position that is calibrated with respect to the laser may be used to determine whether a deviation or mismatch is actually due to a subject position deviating from a target position.

If it is determined that the subject position corresponds to the target position (e.g., after an alignment procedure) and a deviation or mismatch between the projections is observed, it may be determined that the registration of the AR device requires adjustment. In response to determining that the registration requires adjustment, an adjustment may automatically be triggered or a user may be alerted.

The method of the present disclosure may comprise, by means of a calibration procedure, determining an initial first component, an initial second component, and an initial third component of the position of the target point in AR device coordinates, storing the initial first component, the initial second component, and the initial third component of the position of the target point in AR device coordinates as (the) previously determined and stored first component, second component, and third component, respectively. Optionally, the calibration procedure may comprise using, in addition to images obtained by means of the camera of the AR system, measurement data from other devices, the measurement data being data obtained prior to and/or during the calibration procedure.

Determining the initial first component, second component, and/or third component may be performed in a manner similar to the above-described methods described in the context of adjusting the registration. However, this may be computationally expensive and/or inaccurate when starting with no prior registration. Other method steps than the ones described in the context of adjusting the registration may be used for determining the initial first, second, and/or third components other method steps. These method steps may be selected for an initial registration at lower accuracy than the adjusting of the registration, e.g., so as to form a basis for adjusting the registration with the method steps described above in the context of adjusting the registration. Alternatively, the method steps may be selected for a high-precision initial registration based on method steps that yield the same or higher accuracy than the adjusting of the registration. In particular, an initial registration may comprise using image data obtained using imaging modalities that are not or only infrequently used for the adjusting of the registration, for example X-ray imaging and/or image processing steps that are not or only infrequently used for the adjusting of the registration, for example registering live surface image data with live 2D image data. It can be seen from the above that it may be very advantageous from an accuracy and/or a resource point of view to perform an initial calibration procedure, particularly leveraging different and/or additional data compared to the adjusting steps.

Proceeding with the initial components may, for example, be performed as described above in the context of using the previously determined and stored components. This is reflected by the feature that the initial components are stored as the previously determined and stored components.

Moreover, for the sake of completeness, it is noted that the method may comprise, prior to acquiring the first image, placing a subject in such a manner that the first laser beam, in particular, additional of the second and/or third laser beam are projected onto the subject. In particular, after placing the subject, an alignment of the subject may be performed prior to acquiring the first image, in particular, an alignment such that the subject has a fixed position relative to the target point. Such an alignment may be performed using known methods.

The present disclosure also provides an augmented reality, AR, system comprising an augmented reality, AR, device configured to overlay a virtual image with a field of view of the AR device, and a camera being part of the AR device and/or connected to the AR device. The AR system is configured to carry out any of the methods of the present disclosure.

The AR device may comprise AR glasses, in particular, AR glasses comprising the camera. The AR device, particularly the AR glasses, may comprise a see-through display and the virtual image may be overlaid onto the reality as observed through the see-though display. Alternatively or in addition, the virtual image may be overlayed with live-image data of the field of view of the AR device. Alternatively or in addition to AR glasses, the AR device may comprise a hand-held device, e.g., a tablet.

The camera may be part of or connected to the AR device. As an example, the camera may be incorporated in the AR glasses. The camera may be configured to obtain the first and/or second image, in particular, may be configured to acquire images continuously or at predetermined times and/or intervals. The latter allows for monitoring the registration over time and adjusting it when needed.

The AR system may comprise, separately and/or as part of the AR device, processing means configured to carry out the processing steps of the method of the present disclosure.

The present disclosure also provides a medical system comprising the AR system of the present disclosure and further comprising a first laser configured to provide the first laser beam in the first plane. Optionally, the medical system may further comprise a second laser configured to provide the second laser beam in the second plane and/or a third laser configured to provide the third laser beam in the third plane.

The medical system may be configured such that, after setup for operation, the first laser, second laser, and third laser are arranged in a predetermined and fixed position and orientation, particularly, in such a manner as to provide laser beams in three orthogonal planes intersecting at the target point or at a point having a fixed spatial relation with respect to the target point. In particular, the planes may comprise vertical and horizontal planes. As mentioned above, each laser may have a fan laser geometry that provides a laser beam expanded in the respective plane and/or that scans the laser beam along a line in the respective plane.

The medical system of the present disclosure may further comprise a treatment machine, in particular a radiation treatment apparatus comprising a treatment beam source and a patient support unit, and the lasers and the treatment machine may be configured and/or arranged such that the target point, in particular the intersection point of the first, second, and third planes, coincides with the isocenter of the treatment beam source.

Thus, the laser beams precisely mark the position of the isocenter and their projection onto the subject and/or onto other objects can be used to reconstruct the isocenter and, accordingly, to align the subject and/or the other objects, e.g., imaging devices and/or instruments and/or a subject support unit, with respect to the isocenter. Using the isocenter as a target point for the adjustment of the registration allows for high accuracy, as all elements can be aligned and/or registered with respect to one common point, thereby avoiding inaccuracies that may arise when using more indirect alignment and/or registration.

The present disclosure also provides a computer program which, when running on at least one processor of at least one computer or when loaded into at least one memory of at least one computer, causes the at least one computer to carry out and/or control any or all steps of the method according to the present disclosure. The present disclosure alternatively or additionally provides a (physical, for example electrical, for example technically generated) signal wave, for example a digital signal wave, carrying information which represents the program, for example the aforementioned program, which for example comprises code means which are adapted to carry out and/or control any or all steps of the method according to the present disclosure.

A computer program stored on a disc is a data file, and when the file is read out and transmitted it becomes a data stream for example in the form of a (physical, for example electrical, for example technically generated) signal. The signal can be implemented as the signal wave which is described herein. For example, the signal, for example the signal wave is constituted to be transmitted via a computer network, for example LAN, WLAN, WAN, for example the internet. The present disclosure therefore may alternatively or additionally relate to a data stream representative of the aforementioned program.

The present disclosure also provides a, e.g., non-transitory computer-readable medium, program storage medium on which the program. The present disclosure also provides at least one computer, comprising at least one processor and at least one memory, wherein the program according to the present disclosure is running on the processor or is loaded into the memory, or wherein the at least one computer comprises the computer-readable program storage medium according to the present disclosure.

In other words, as an example, the present disclosure may provide a computer program product comprising instructions to cause the augmented reality system and/or the medical system of the present disclosure to execute any of the steps of the method according to the present disclosure, particularly of the method claims. The present disclosure may also provide a computer-readable medium having stored thereon the computer program product.

For example, the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention may instead be directed as applicable to image registration for the sake of proper relative arrangement of a subject and one or more objects e.g., pieces of equipment. For this reason alone, no surgical or therapeutic activity and in particular no surgical or therapeutic step is necessitated or implied by carrying out the invention.

The present disclosure also relates to the use of the method and/or system of the present disclosure for performing a positioning and/or alignment of a subject and various object, e.g., pieces of equipment.

The features and advantages outlined above in the context of the method similarly apply to the AR system, medical system, computer program, program storage medium, computer, signal wave, data stream and use of the medical system described in the present disclosure.

### Definitions

In this section, definitions for specific terminology used in this disclosure are offered which also form part of the present disclosure.

### Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Registering

The n-dimensional image of an object is registered when the spatial location of each point of an actual object within a space, for example a body part in an operating theatre, is assigned an image data point of an image (CT, MR, etc.) stored in a navigation system.

### Image Registration

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements. Furthermore, images that have been created for use in augmented reality systems to overlay them with the environment may be subject to image registration with images representing the environment.

### Marker

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

### Marker Device

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

### Marker Holder

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

### Pointer

A pointer is a rod which comprises one or more-advantageously, two-markers fastened to it and which can be used to measure off individual co-ordinates, for example spatial co-ordinates (i.e. three-dimensional co-ordinates), on a part of the body, wherein a user guides the pointer (for example, a part of the pointer which has a defined and advantageously fixed position with respect to the at least one marker attached to the pointer) to the position corresponding to the co-ordinates, such that the position of the pointer can be determined by using a surgical navigation system to detect the marker on the pointer. The relative location between the markers of the pointer and the part of the pointer used to measure off co-ordinates (for example, the tip of the pointer) is for example known. The surgical navigation system then enables the location (of the three-dimensional co-ordinates) to be assigned to a predetermined body structure, wherein the assignment can be made automatically or by user intervention.

### Reference Star

A “reference star” refers to a device with a number of markers, advantageously three markers, attached to it, wherein the markers are (for example detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star for example features a way of being attached to the object (for example, a clamp and/or a thread) and/or a holding element which ensures a distance between the markers and the object (for example in order to assist the visibility of the markers to a marker detection device) and/or marker holders which are mechanically connected to the holding element and which the markers can be attached to.

### Navigation System

The present disclosure may be applied in the context of a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

### Surgical Navigation System

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

### Landmarks

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

### Imaging Geometry

The information on the imaging geometry preferably comprises information which allows the analysis image (x-ray image) to be calculated, given a known relative position between the imaging geometry analysis apparatus and the analysis object (anatomical body part) to be analysed by x-ray radiation, if the analysis object which is to be analysed is known, wherein “known” means that the spatial geometry (size and shape) of the analysis object is known. This means for example that three-dimensional, “spatially resolved” information concerning the interaction between the analysis object (anatomical body part) and the analysis radiation (x-ray radiation) is known, wherein “interaction” means for example that the analysis radiation is blocked or partially or completely allowed to pass by the analysis object. The location and in particular orientation of the imaging geometry is for example defined by the position of the x-ray device, for example by the position of the x-ray source and the x-ray detector and/or for example by the position of the multiplicity (manifold) of x-ray beams which pass through the analysis object and are detected by the x-ray detector. The imaging geometry for example describes the position (i.e. the location and in particular the orientation) and the shape (for example, a conical shape exhibiting a specific angle of inclination) of said multiplicity (manifold). The position can for example be represented by the position of an x-ray beam which passes through the centre of said multiplicity or by the position of a geometric object (such as a truncated cone) which represents the multiplicity (manifold) of x-ray beams. Information concerning the above-mentioned interaction is preferably known in three dimensions, for example from a three-dimensional CT, and describes the interaction in a spatially resolved way for points and/or regions of the analysis object, for example for all of the points and/or regions of the analysis object. Knowledge of the imaging geometry for example allows the location of a source of the radiation (for example, an x-ray source) to be calculated relative to an image plane (for example, the plane of an x-ray detector). With respect to the connection between three-dimensional analysis objects and two-dimensional analysis images as defined by the imaging geometry, reference is made for example to the following publications:
1. “An Efficient and Accurate Camera Calibration Technique for 3D Machine Vision”, Roger Y. Tsai, Proceedings of the IEEE Conference on Computer Vision and Pattern Recognition. Miami Beach, Florida, 1986, pages 364-374
2. “A Versatile Camera Calibration Technique for High-Accuracy 3D Machine Vision Metrology Using Off-the-Shelf TV Cameras and Lenses”, Roger Y. Tsai, IEEE Journal of Robotics and Automation, Volume RA-3, No. 4, August 1987, pages 323-344.
3. “Fluoroscopic X-ray Image Processing and Registration for Computer-Aided Orthopedic Surgery”, Ziv Yaniv
4. EP 08 156 293.6
5. US 61/054,187

### Shape Representatives

Shape representatives represent a characteristic aspect of the shape of an anatomical structure. Examples of shape representatives include straight lines, planes and geometric figures. Geometric figures can be one-dimensional such as for example axes or circular arcs, two-dimensional such as for example polygons and circles, or three-dimensional such as for example cuboids, cylinders and spheres. The relative position between the shape representatives can be described in reference systems, for example by co-ordinates or vectors, or can be described by geometric variables such as for example length, angle, area, volume and proportions. The characteristic aspects which are represented by the shape representatives are for example symmetry properties which are represented for example by a plane of symmetry. Another example of a characteristic aspect is the direction of extension of the anatomical structure, which is for example represented by a longitudinal axis. Another example of a characteristic aspect is the cross-sectional shape of an anatomical structure, which is for example represented by an ellipse. Another example of a characteristic aspect is the surface shape of a part of the anatomical structure, which is for example represented by a plane or a hemisphere. For example, the characteristic aspect constitutes an abstraction of the actual shape or an abstraction of a property of the actual shape (such as for example its symmetry properties or longitudinal extension). The shape representative for example represents this abstraction.

### Referencing

Determining the position is referred to as referencing if it implies informing a navigation system of said position in a reference system of the navigation system.

### Atlas/Atlas Segmentation

Preferably, atlas data is acquired which describes (for example defines, more particularly represents and/or is) a general three-dimensional shape of the anatomical body part. The atlas data therefore represents an atlas of the anatomical body part. An atlas typically consists of a plurality of generic models of objects, wherein the generic models of the objects together form a complex structure. For example, the atlas constitutes a statistical model of a patient's body (for example, a part of the body) which has been generated from anatomic information gathered from a plurality of human bodies, for example from medical image data containing images of such human bodies. In principle, the atlas data therefore represents the result of a statistical analysis of such medical image data for a plurality of human bodies. This result can be output as an image—the atlas data therefore contains or is comparable to medical image data. Such a comparison can be carried out for example by applying an image fusion algorithm which conducts an image fusion between the atlas data and the medical image data. The result of the comparison can be a measure of similarity between the atlas data and the medical image data. The atlas data comprises image information (for example, positional image information) which can be matched (for example by applying an elastic or rigid image fusion algorithm) for example to image information (for example, positional image information) contained in medical image data so as to for example compare the atlas data to the medical image data in order to determine the position of anatomical structures in the medical image data which correspond to anatomical structures defined by the atlas data.

The human bodies, the anatomy of which serves as an input for generating the atlas data, advantageously share a common feature such as at least one of gender, age, ethnicity, body measurements (e.g. size and/or mass) and pathologic state. The anatomic information describes for example the anatomy of the human bodies and is extracted for example from medical image information about the human bodies. The atlas of a femur, for example, can comprise the head, the neck, the body, the greater trochanter, the lesser trochanter and the lower extremity as objects which together make up the complete structure. The atlas of a brain, for example, can comprise the telencephalon, the cerebellum, the diencephalon, the pons, the mesencephalon and the medulla as the objects which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

### Analytical Devices

The movements of the treatment body parts are for example due to movements which are referred to in the following as “vital movements”. Reference is also made in this respect to EP 2 189 943 A1 and EP 2 189 940 A1, also published as US 2010/0125195 A1 and US 2010/0160836 A1, respectively, which discuss these vital movements in detail. In order to determine the position of the treatment body parts, analytical devices such as x-ray devices, CT devices or MRT devices are used to generate analytical images (such as x-ray images or MRT images) of the body. For example, analytical devices are constituted to perform medical imaging methods. Analytical devices for example use medical imaging methods and are for example devices for analysing a patient's body, for instance by using waves and/or radiation and/or energy beams, for example electromagnetic waves and/or radiation, ultrasound waves and/or particles beams. Analytical devices are for example devices which generate images (for example, two-dimensional or three-dimensional images) of the patient's body (and for example of internal structures and/or anatomical parts of the patient's body) by analysing the body. Analytical devices are for example used in medical diagnosis, for example in radiology. However, it can be difficult to identify the treatment body part within the analytical image. It can for example be easier to identify an indicator body part which correlates with changes in the position of the treatment body part and for example the movement of the treatment body part. Tracking an indicator body part thus allows a movement of the treatment body part to be tracked on the basis of a known correlation between the changes in the position (for example the movements) of the indicator body part and the changes in the position (for example the movements) of the treatment body part. As an alternative to or in addition to tracking indicator body parts, marker devices (which can be used as an indicator and thus referred to as “marker indicators”) can be tracked using marker detection devices. The position of the marker indicators has a known (predetermined) correlation with (for example, a fixed relative position relative to) the position of indicator structures (such as the thoracic wall, for example true ribs or false ribs, or the diaphragm or intestinal walls, etc.) which for example change their position due to vital movements.

### Treatment Beam

The present invention relates to the field of controlling a treatment beam. The treatment beam treats body parts which are to be treated and which are referred to in the following as “treatment body parts”. These body parts are for example parts of a patient's body, i.e. anatomical body parts.

The present invention relates to the field of medicine and for example to the use of beams, such as radiation beams, to treat parts of a patient's body, which are therefore also referred to as treatment beams. A treatment beam treats body parts which are to be treated and which are referred to in the following as “treatment body parts”. These body parts are for example parts of a patient's body, i.e. anatomical body parts. Ionising radiation is for example used for the purpose of treatment. For example, the treatment beam comprises or consists of ionising radiation. The ionising radiation comprises or consists of particles (for example, sub-atomic particles or ions) or electromagnetic waves which are energetic enough to detach electrons from atoms or molecules and so ionise them. Examples of such ionising radiation include x-rays, high-energy particles (high-energy particle beams) and/or ionising radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumour are treated using ionising radiation. The tumour is then an example of a treatment body part.

The treatment beam is preferably controlled such that it passes through the treatment body part. However, the treatment beam can have a negative effect on body parts outside the treatment body part. These body parts are referred to here as “outside body parts”. Generally, a treatment beam has to pass through outside body parts in order to reach and so pass through the treatment body part.

Reference is also made in this respect to the following web pages:

| | |
|---|---|
| http://www.elekta.com/healthcare_us_elekta_vmat.php | and |
| http://www.varian.com/us/oncology/treatments/treatment_techniques/rapidarc. | |

### Arrangement of Treatment Beams

A treatment body part can be treated by one or more treatment beams issued from one or more directions at one or more times. The treatment by means of the at least one treatment beam thus follows a particular spatial and temporal pattern. The term “beam arrangement” is then used to cover the spatial and temporal features of the treatment by means of the at least one treatment beam. The beam arrangement is an arrangement of at least one treatment beam.

The “beam positions” describe the positions of the treatment beams of the beam arrangement. The arrangement of beam positions is referred to as the positional arrangement. A beam position is preferably defined by the beam direction and additional information which allows a specific location, for example in three-dimensional space, to be assigned to the treatment beam, for example information about its co-ordinates in a defined co-ordinate system. The specific location is a point, preferably a point on a straight line. This line is then referred to as a “beam line” and extends in the beam direction, for example along the central axis of the treatment beam. The defined co-ordinate system is preferably defined relative to the treatment device or relative to at least a part of the patient's body. The positional arrangement comprises and for example consists of at least one beam position, for example a discrete set of beam positions (for example, two or more different beam positions), or a continuous multiplicity (manifold) of beam positions.

For example, one or more treatment beams adopt(s) the treatment beam position(s) defined by the positional arrangement simultaneously or sequentially during treatment (for example sequentially if there is only one beam source to emit a treatment beam). If there are several beam sources, it is also possible for at least a subset of the beam positions to be adopted simultaneously by treatment beams during the treatment. For example, one or more subsets of the treatment beams can adopt the beam positions of the positional arrangement in accordance with a predefined sequence. A subset of treatment beams comprises one or more treatment beams. The complete set of treatment beams which comprises one or more treatment beams which adopt(s) all the beam positions defined by the positional arrangement is then the beam arrangement.

### Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia. The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

### Mapping

Mapping describes a transformation (for example, linear transformation) of an element (for example, a pixel or voxel), for example the position of an element, of a first data set in a first coordinate system to an element (for example, a pixel or voxel), for example the position of an element, of a second data set in a second coordinate system (which may have a basis which is different from the basis of the first coordinate system). In one embodiment, the mapping is determined by comparing (for example, matching) the color values (for example grey values) of the respective elements by means of an elastic or rigid fusion algorithm. The mapping is embodied for example by a transformation matrix (such as a matrix defining an affine transformation).

### Elastic Fusion, Image Fusion/Morphing, Rigid

Image fusion can be elastic image fusion or rigid image fusion. In the case of rigid image fusion, the relative position between the pixels of a 2D image and/or voxels of a 3D image is fixed, while in the case of elastic image fusion, the relative positions are allowed to change.

In this application, the term “image morphing” is also used as an alternative to the term “elastic image fusion”, but with the same meaning.

Elastic fusion transformations (for example, elastic image fusion transformations) are for example designed to enable a seamless transition from one dataset (for example a first dataset such as for example a first image) to another dataset (for example a second dataset such as for example a second image). The transformation is for example designed such that one of the first and second datasets (images) is deformed, for example in such a way that corresponding structures (for example, corresponding image elements) are arranged at the same position as in the other of the first and second images. The deformed (transformed) image which is transformed from one of the first and second images is for example as similar as possible to the other of the first and second images. Preferably, (numerical) optimisation algorithms are applied in order to find the transformation which results in an optimum degree of similarity. The degree of similarity is preferably measured by way of a measure of similarity (also referred to in the following as a “similarity measure”). The parameters of the optimisation algorithm are for example vectors of a deformation field. These vectors are determined by the optimisation algorithm in such a way as to result in an optimum degree of similarity. Thus, the optimum degree of similarity represents a condition, for example a constraint, for the optimisation algorithm. The bases of the vectors lie for example at voxel positions of one of the first and second images which is to be transformed, and the tips of the vectors lie at the corresponding voxel positions in the transformed image. A plurality of these vectors is preferably provided, for instance more than twenty or a hundred or a thousand or ten thousand, etc. Preferably, there are (other) constraints on the transformation (deformation), for example in order to avoid pathological deformations (for instance, all the voxels being shifted to the same position by the transformation). These constraints include for example the constraint that the transformation is regular, which for example means that a Jacobian determinant calculated from a matrix of the deformation field (for example, the vector field) is larger than zero, and also the constraint that the transformed (deformed) image is not self-intersecting and for example that the transformed (deformed) image does not comprise faults and/or ruptures. The constraints include for example the constraint that if a regular grid is transformed simultaneously with the image and in a corresponding manner, the grid is not allowed to interfold at any of its locations. The optimising problem is for example solved iteratively, for example by means of an optimisation algorithm which is for example a first-order optimisation algorithm, such as a gradient descent algorithm. Other examples of optimisation algorithms include optimisation algorithms which do not use derivations, such as the downhill simplex algorithm, or algorithms which use higher-order derivatives such as Newton-like algorithms. The optimisation algorithm preferably performs a local optimisation. If there is a plurality of local optima, global algorithms such as simulated annealing or generic algorithms can be used. In the case of linear optimisation problems, the simplex method can for instance be used.

In the steps of the optimisation algorithms, the voxels are for example shifted by a magnitude in a direction such that the degree of similarity is increased. This magnitude is preferably less than a predefined limit, for instance less than one tenth or one hundredth or one thousandth of the diameter of the image, and for example about equal to or less than the distance between neighbouring voxels. Large deformations can be implemented, for example due to a high number of (iteration) steps.

The determined elastic fusion transformation can for example be used to determine a degree of similarity (or similarity measure, see above) between the first and second datasets (first and second images). To this end, the deviation between the elastic fusion transformation and an identity transformation is determined. The degree of deviation can for instance be calculated by determining the difference between the determinant of the elastic fusion transformation and the identity transformation. The higher the deviation, the lower the similarity, hence the degree of deviation can be used to determine a measure of similarity.

A measure of similarity can for example be determined on the basis of a determined correlation between the first and second datasets.

### Neutral Bone Position

The neutral position between two bones is a known position in the field of medicine and depends on the type of joint. In the hip joint as an example, the neutral position is the position in which the mechanical axis of the femur lies in a sagittal plane, for instance parallel to the midsagittal plane, and the mechanical axis and posterior condylar line of the femur describe a plane which is parallel to the frontal plane of the pelvis. The posterior condylar line connects the most posterior and distal femoral points. It might, however, not be possible to acquire or sample those points, for example during surgery. In this case, an option is to acquire the ankle epicondyle piriformis (AEP) plane defined by a piriformis point (the proximal point of the femur shaft axis), the center of the epicondyle axis and an ankle point of the flexed leg. A direction orthogonal to this AEP plane corresponds to the direction of the posterior condylar axis and thus forms, together with the mechanical axis, a plane which is parallel to the frontal plane of the pelvis in the neutral position.

The neutral position between two bones typically is the origin relative to which a range of motion is defined. This neutral position is achieved for a particular joint orientation of the joint between the two bones.

### Fixed (Relative) Position

A fixed position, which is also referred to as fixed relative position, in this document means that two objects which are in a fixed position have a relative position which does not change unless this change is explicitly and intentionally initiated. A fixed position is in particular given if a force or torque above a predetermined threshold has to be applied in order to change the position. This threshold might be 10 N or 10 Nm. In particular, the position of a sensor device remains fixed relative to a target while the target is registered or two targets are moved relative to each other. A fixed position can for example be achieved by rigidly attaching one object to another. The spatial location, which is a part of the position, can in particular be described just by a distance (between two objects) or just by the direction of a vector (which links two objects). The alignment, which is another part of the position, can in particular be described by just the relative angle of orientation (between the two objects).

### Medical Workflow

A medical workflow comprises a plurality of workflow steps performed during a medical treatment and/or a medical diagnosis. The workflow steps are typically, but not necessarily performed in a predetermined order. Each workflow step for example means a particular task, which might be a single action or a set of actions. Examples of workflow steps are capturing a medical image, positioning a patient, attaching a marker, performing a resection, moving a joint, placing an implant and the like.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIGS. 1a and 1b illustrate schematically and not to scale an oblique view of a medical system of the present disclosure and an enlarged view of laser beam projections onto a subject;

FIG. 2 shows schematically and not to scale an AR system of the present disclosure;

FIG. 3 illustrates a simplified view of a first and a second image; and

FIG. 4 is a schematic illustration of a method of the present disclosure.

### DESCRIPTION OF EMBODIMENTS

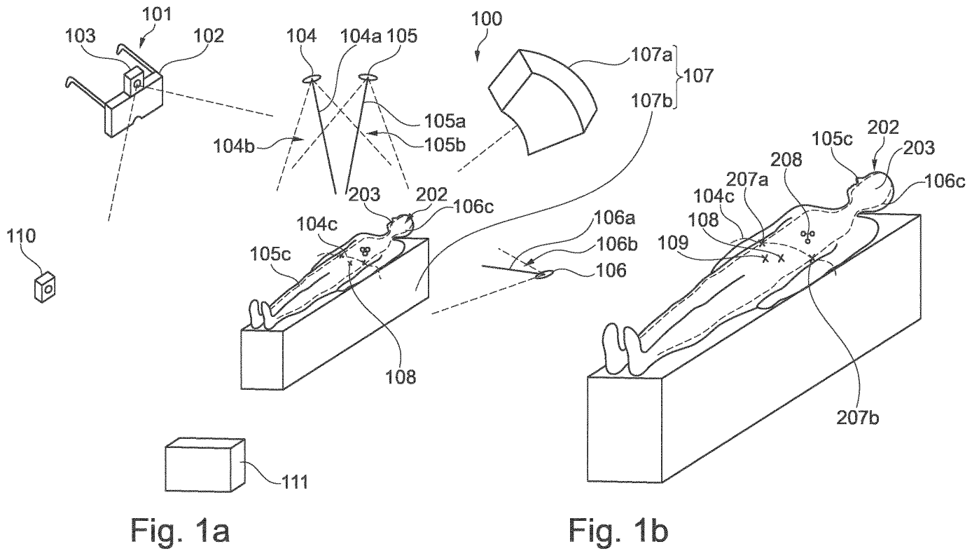

FIG. 1a shows an exemplary augmented reality, AR, system 101 according to the present disclosure. The AR system may be part of a medical system 100 of the present disclosure.

The AR system comprises an AR device 102 and a camera 103. The AR device, in the present example, is configured as AR glasses comprising the camera. However, alternatively, the camera may also be provided separate from and connected to the AR device by means of a data connection.

As illustrated in FIG. 1a, the medical system may comprise a first laser 104 configured to provide a first laser beam 104a in a first plane 104b, a second laser 105 configured to provide a second laser beam 105a in a second plane 105b, and a third laser 106 configured to provide a third laser beam 106a in a third plane 106b.

In operation, the lasers are arranged in fixed positions and orientations. In this example, the first laser and second laser are arranged and configured such that the first plane 104b and the second plane 105b are vertically arranged planes that are orthogonal and intersect in a line that comprises the target point 108. The target point will be explained in more detail below. The third laser is arranged and configured such that the third plane 106b is a horizontally arranged plane that intersects with the first plane 104b and the second plane 105b in the target point.

Other arrangements of the lasers and planes are, however, conceivable. For example, the planes may be orthogonal planes, yet may not be horizontally and vertically arranged. The planes also need not necessarily be orthogonal planes. The planes need not necessarily intersect in one point. If the planes do intersect in one point, this point need not necessarily correspond to the target point but may be a point having a known spatial relation with respect to the target point.

As shown in FIG. 1a, the medical system may comprise a treatment machine 107, for example comprising a treatment beam source 107a and a patient support unit 107b. The medical system may optionally comprise one or more imaging means, for example an additional camera 110, e.g., a surface camera, and/or other imaging means 111, e.g., CT, MRI, and/or X-ray imaging means.

FIG. 1a also shows the surface 202 of a subject 203, in this case a person, placed on the patient support unit 107b. The subject is not part of the system.

Projections of the laser beams 104a, 105a, and 106a onto the surface of the subject are shown as dashed lines 104c, 105c, and 106c, respectively. It is apparent from the oblique view onto the subject as shown in FIG. 1, that, none of the lines are entirely straight. This is due to the subject's surface being curved.

FIG. 1a also shows crossings of the projections. The crossing of projections 104c and 105c is designated with the reference sign 207a, the crossing of projections 104c and 106c is designated with the reference sign 207b.

FIG. 1b shows an enlarged view of the subject and the projections. Merely for the sake of illustration, an optional marker 208 attached to the surface of the subject is shown in FIG. 1b.

An isocenter 109 of the treatment beam source 107a is indicated in FIG. 1b. The isocenter may coincide with the intersection point of the three planes 104b, 105b, and 106b when the treatment beam source is arranged for intended operation. Moreover, the isocenter 109 may coincide with the target point 108 when the subject is arranged in a target position.

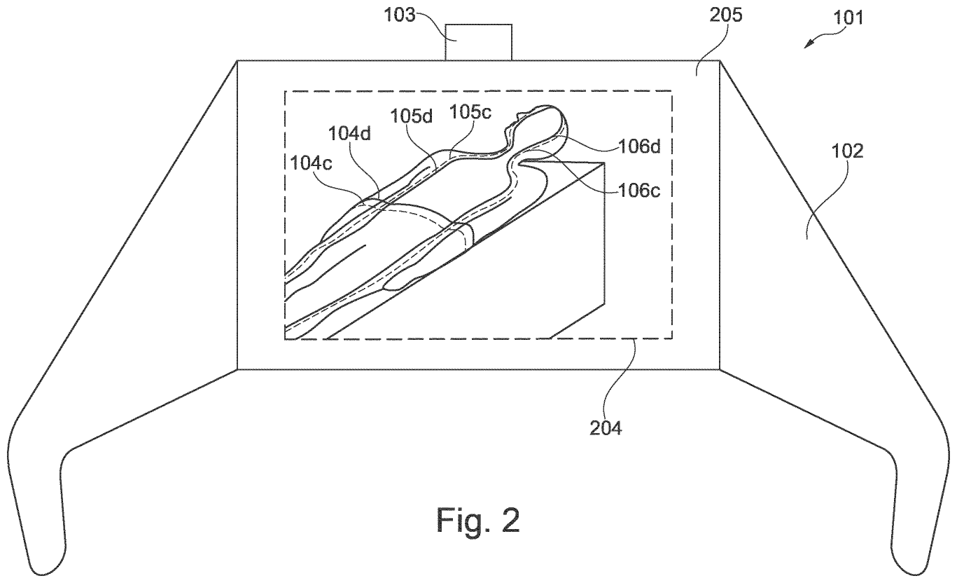

FIG. 2 shows an illustration of an AR system 101 according to the present disclosure comprising an AR device in the form of AR glasses 102 that comprise a camera 103.

Shown in the field of view 205 of the AR glasses is a virtual image 204 overlaid with the field of view. In the present example, the virtual image at least comprises a visualization of expected projections 104d, 105d, and 106d, which is overlaid with the live view, via a camera or through a see-through display, of the subject and the actual projections 104c, 105c, and 106c.

In FIG. 2, there is a mismatch between the expected and the actual projections. This may have two causes, i.e., misalignment of the subject as compared to the alignment on which the expected projections were modelled and inaccurate registration of the AR device. In such a case, the user of the AR device may trigger verification of the subject alignment, for example, using camera 110 and/or imaging means 111. If the subject alignment has been verified or accounted for and a mismatch still remains, this would indicate that the registration is inaccurate.

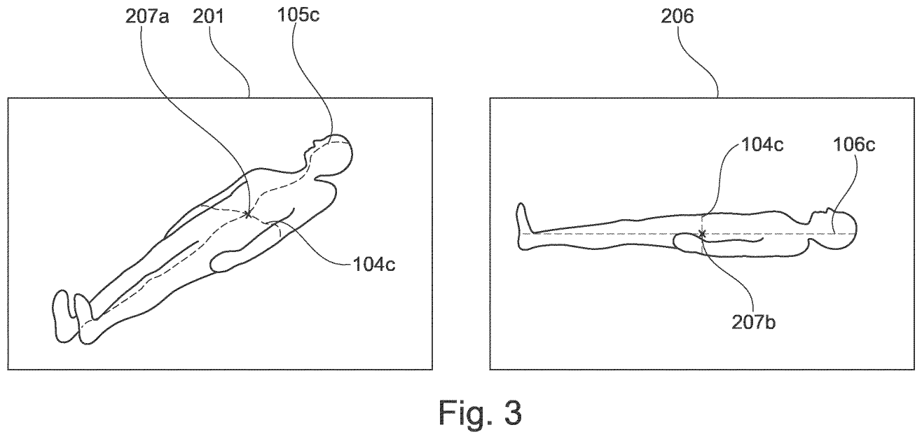

In FIG. 3, an exemplary first image 201 and an exemplary second image 206 are shown to illustrate the case when the first image 201 depicts the first projection 104c and the second projection 105c, but does not depict the third projection 106c, such that the third component is derived (at least) from an additional second image 206, which depicts the third projection 106c. In this example, the second image also depicts the first projection 104c, but does not depict the second projection 105c. Alternatively or in addition, the second image might depict, in addition to the third projection 106c, the second projection 105c.

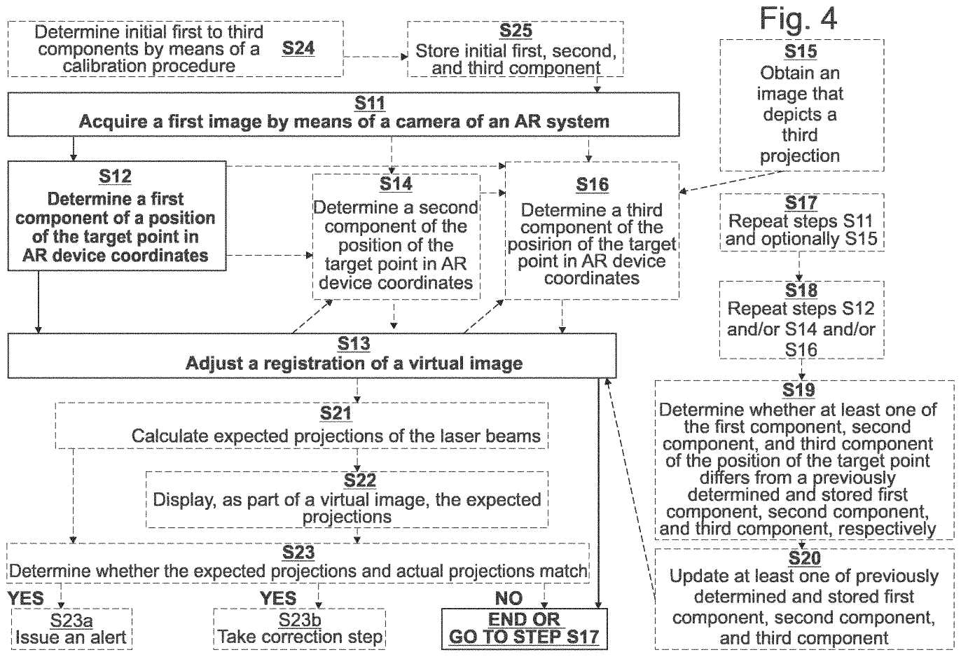

FIG. 4 illustrates an exemplary method according to the present disclosure. The method may be performed using an AR system or medical system comprising the AR system according to the present disclosure, particularly the AR system or medical system described in the context of FIGS. 1a and 2, or any other suitable system.

However, for the sake of illustration, reference signs of FIGS. 1a and 2 will be used in the following description.

The method comprises a step S11 of acquiring a first image by means of a camera 103 of an AR system 101.

The first image may depict a first projection 104c of a first laser beam 104a onto a surface of a subject, the first projection 104c being in the shape of a first line representing the intersection of a first plane 104b with the surface 202 of the subject 203. The first plane 104b is arranged such that it comprises or has a fixed spatial relation with respect to a target point 108. In addition, a second projection 105c of a second laser beam 105a and/or a third projection 106b of a third laser beam 106a may be depicted.

The method further comprises a step S12 of determining a first component of a position of the target point 108 in AR device coordinates, for example, an x-, y-, or z-coordinate of the target point. This is performed based on the shape and/or position of the first line in the first image. For example, a set of candidate shapes and/or positions may be available for different values of the first component and a matching may be performed to determine the current value. 3D image date, information from markers, or information from the other projections in the image may optionally be used in addition to the shape and/or position of the first projection.

The method further comprises a step S13 of adjusting a registration of a virtual image at least based on the first component. That is, a currently stored value for the stored first component may be updated on the basis of the determination. The virtual image may be registered relative to the target point, such that the registration may be adjusted when the first component of the target point is updated.

Similar steps may optionally be performed for the other components of the position of the target point in AR device coordinates. For example, in step S14, a second component of the position of the target point in AR device coordinates may be determined and/or in step S16, a third component of the position of the target point in AR device coordinates may be determined, e.g., the other two coordinates of the target point.

In case steps S14 and/or S16 are performed, step S13 of adjusting the registration of the virtual image is performed based on the first component and the second and/or third component.

In order to determine the third component, the method may comprise a step S15 of obtaining an image that depicts a third projection, which may be the first image 201 or a second image 206, in which case the obtaining of the image comprises acquiring the second image particularly at the time of acquiring the first image.

Optionally, the steps of acquiring/obtaining the first and optionally second image may be performed repeatedly (step S17), e.g., continuously or at predetermined times or time intervals. Similarly, the determining of the first, second and/or third component may be performed repeatedly (step S18), e.g., continuously or at predetermined times or time intervals.

The method may then comprise the step S19 of determining whether at least one of the first component, the second component, and the third component of the position of the target point differs from a previously determined and stored first component, second component, and third component, respectively.

In response to determining that this is the case, in step S20, at least one of the previously determined and stored first component, second component, and third component may be updated in accordance with the most recent component.

In optional step S21, expected projections 104d, 105d, and 106d of the laser beams may be calculated and in step S22, the AR device may display, as part of a virtual image, the expected projections, e.g., as shown in FIG. 2. Optionally, in step S23, it may be determined from an image obtained by the camera 103 whether the expected projections and actual projections match. If this is not the case, an alert may be issued in step S23a and/or correction steps may be taken in step S23b.

In optional step S24, initial first to third components may be determined by means of a calibration procedure, prior to the steps S11 et seq., and in step S25 the initial first to third components may be stored. The stored values may be used, as an example, for any of the subsequent determination steps.

Another exemplary method is described below.

A first laser beam and a second laser beam and optionally a third laser beam may be projected into a treatment room. The first and the second laser beam and optionally the third laser beam may be calibrated to cross in the isocenter of a treatment beam source, e.g., a LINAC. A phantom may be arranged and aligned with the lasers, the phantom having a marker with a known position relative to the isocenter. An initial registration may be determined between the augmented reality device and the treatment room isocenter. The phantom may then be removed, and a patient may be position in the area of the isocenter. Projections of the laser beams will appear on the surface of the patient as lines having a profile. At least one image depicting the projections may be acquired with one or more cameras and based on the shape of the projections in the image at least one of the x, y-, and z-coordinate can be obtained. The coordinate(s) may be used to correct a current registration of the augmented reality device. Thus, image data may be overlaid on the field of view of the augmented reality device such that the room isocenter and the image isocenter coordinate systems align.

While the invention has been illustrated and described in detail in the drawings and foregoing description, such illustration and description are to be considered exemplary and not restrictive. The invention is not limited to the disclosed embodiments. In view of the foregoing description and drawings it will be evident to a person skilled in the art that various modifications may be made within the scope of the invention, as defined by the claims.

1. A method for registration of a virtual image in an augmented reality, AR, system comprising an AR device, the method comprising:
acquiring a first image by a camera of the AR system, wherein the first image depicts a first projection of a first laser beam onto a surface of a subject, the first projection being in the shape of a first line representing the intersection of a first plane with the surface of the subject, the first plane arranged such that it comprises or has a fixed spatial relation with respect to a target point;
determining, based at least on a shape and/or position of the first line in the first image, a first component of a position of the target point in AR device coordinates; and
adjusting a registration of a virtual image, which is to be overlaid with the field of view of the AR device, based on the first component of the position of the target point in AR device coordinates.

2. The method of claim 1,
wherein the first image further depicts a second projection of a second laser beam onto the surface of the subject, the second projection being in the shape of a second line representing the intersection of a second plane with the surface of the subject;
wherein an intersection of the first plane and the second plane comprises the target point or has a fixed spatial relation with respect to the target point;
further comprising determining, based at least on a shape and/or position of the second line in the first image, and optionally also based on the shape and/or position of the first line in the first image, a second component of the position of the target point in AR device coordinates; and
wherein the adjusting of the registration of the virtual image is performed based on the first component and the second component of the target point in AR device coordinates.

3. The method of claim 1, further comprising determining a third component of the position of the target point in the AR device coordinates,
wherein adjusting the registration of the virtual image is performed based on the first component, the second component, and the third component of the position of the target point in AR device coordinates,
the method further comprising:
obtaining an image that depicts a third projection of a third laser beam onto the surface of the subject, wherein the third projection is in the shape of a third line representing the intersection of a third plane with the surface of the subject, the third plane being arranged such that the intersection of the first plane, the third plane, and optionally the second plane, comprises the target point or has a fixed spatial relation with respect to the target point; and
determining, based at least on a shape and/or position of the third line in the image, and optionally also based on the shape and/or position of the first line and/or the second line, the third component of the position of the target point in AR device coordinates,
wherein the image is the first image or a second image acquired by the camera of the AR system and depicting at least one of the first projection and the second projection.

4. The method of claim 1, wherein determining the first component and/or determining the second component and/or determining the third component of the position of the target point in AR device coordinates is further based on a shape of the surface of the subject obtained from 3D image data of the surface.

5. The method of claim 4, wherein determining the first component and/or the second component and/or the third component of the position of the target point in AR device coordinates comprises:
determining, based on the shape of the surface of the subject, a plurality of candidate lines and/or a plurality candidate crossing positions of crossings of candidate lines in the first image and/or in the second image;
matching the first line and/or the second line and/or the third line and/or one or more crossing positions of crossings of the first line and/or the second line and/or the third line in the first image and/or in the second image with the plurality of candidate lines and/or the plurality of candidate crossing positions, so as to obtain one or more matching candidate lines and/or one or more matching candidate crossing positions; and
determining the first component and/or the second component and/or the third component of the position of the target point in AR device coordinates based on the one or more matching candidate lines and/or the one or more matching candidate crossing positions.

6. The method of claim 1, comprising
reconstructing the first plane from the shape of the first line in the first image, wherein determining the first component of the position of the target point in AR device coordinates is based at least on the reconstructed first plane, and/or
reconstructing the second plane from the shape of the second line in the first image, wherein determining the second component of the position of the target point in AR device coordinates is based at least on the reconstructed second plane, and/or
reconstructing the third plane from the shape of the third line in the image, in particular the first image or the second image, wherein determining the third component of the position of the target point in AR device coordinates is based at least on the reconstructed third plane.

7. The method of claim 1,
wherein the reconstructing of the first plane and/or the second plane and/or the third plane is further based on:
a/the shape of the surface of the subject obtained from 3D image data of the surface, and/or
a position of the first line and/or the second line and/or the third line in the first image and/or the second image, and/or
a relative position of the first line with respect to the second line and/or the third line in the first image, and/or
a relative position of the first line and/or the second line with respect to the third line in the second image, and/or
one or more crossing positions of the first line and/or the second line and/or the third line in the first image and/or in the second image.

8. The method of claim 1, wherein determining the first component and/or second component and/or third component of the position of the target point in AR device coordinates is further based on:
additional measurement data, from the camera and/or from an additional camera, and/or image data from a CT or MRI imaging device, and/or gyroscopic tracking data obtained by the augmented reality system and/or inside-out tracking data; and/or
information on the shape of the subject and/or arrangement of the subject relative to the camera and/or the target point; and/or
position information on a marker located on the subject and depicted in the first image and/or second image; and/or
information on an arrangement of the first plane and/or the second plane and/or the third plane, in particular an arrangement relative to each other and/or the subject and/or the camera and/or the target point; and/or
previously determined values of the first component and/or second component and/or third component of the position of the target point in AR device coordinates and/or information on at least one of a previously determined reconstructed first plane and/or second plane and/or third plane; and/or
information obtained by a calibration procedure.

9. The method of claim 1, comprising
continuously or at predetermined times and/or time intervals repeating the steps of acquiring the first image, and optionally obtaining the second image;
determining at least one of the first component, the second component, and the third component of the position of the target point in AR device coordinates;
determining whether at least one of the first component, second component, and third component of the position of the target point in AR device coordinates differs from a previously determined and stored first component, second component, and third component, respectively, of the position of the target point in AR device coordinates; and
in response to determining that this is the case, updating at least one of the previously determined and stored first component, second component, and third component.

10. The method of claim 1, further comprising
calculating, based on measurements performed in advance of placing the subject and based on the target position and orientation of the subject with respect to the target point or based on measurements determining the surface of the subject and the placement of the subject relative to the laser beams when the subject is placed, the expected projections of each of the laser beams, and
the AR device displaying, as part of the virtual image, the expected projections of the laser beams, to allow for a user of the AR device to determine whether the expected projections and the actual projections match, and/or
determining, from an image obtained by the camera, that the projections of each of the laser beams deviate from the expected projection, and, in case that the deviation exceeds a predetermined threshold, outputting an alert and/or taking automatic correction steps.

11. The method of claim 1, further comprising,
determining, by a calibration procedure, an initial first component, an initial second component, and an initial third component of the position of the target point in AR device coordinates, storing the initial first component, the initial second component, and the initial third component of the position of the target point in AR device coordinates as previously determined and stored first component, second component, and third component, respectively,
optionally wherein the calibration procedure comprises using, in addition to images obtained by the camera of the AR system, measurement data from other devices, the measurement data being data obtained prior to and/or during the calibration procedure.

12. An augmented reality, AR, system comprising:
an augmented reality, AR, device configured to overlay a virtual image with a field of view of the AR device; and
a camera being part of the AR device and/or connected to the AR device,
wherein the AR system is configured with stored instructions and at least one processor to execute instructions causing the at least one processor to:
acquire a first image by a camera of the AR system, wherein the first image depicts a first projection of a first laser beam onto a surface of a subject, the first projection being in the shape of a first line representing the intersection of a first plane with the surface of the subject, the first plane arranged such that it comprises or has a fixed spatial relation with respect to a target point;
determine, based at least on a shape and/or position of the first line in the first image, a first component of a position of the target point in AR device coordinates; and
adjust a registration of a virtual image, which is to be overlaid with the field of view of the AR device, based on the first component of the position of the target point in AR device coordinates.

13. A medical system comprising:
an AR system having an augmented reality, AR, device configured to overlay a virtual image with a field of view of the AR device; and
a camera being part of the AR device and/or connected to the AR device,
wherein the AR system is configured with stored instructions and at least one processor to execute the stored instructions and causing the at least one processor to:
acquire a first image by a camera of the AR system, wherein the first image depicts a first projection of a first laser beam onto a surface of a subject, the first projection being in the shape of a first line representing the intersection of a first plane with the surface of the subject, the first plane arranged such that it comprises or has a fixed spatial relation with respect to a target point;
determine, based at least on a shape and/or position of the first line in the first image a first component of a position of the target point in AR device coordinates; and
adjust a registration of a virtual image, which is to be overlaid with the field of view of the AR device, based on the first component of the position of the target point in AR device coordinates;
and further including a first laser configured to provide the first laser beam in the first plane, and optionally a second laser configured to provide the second laser beam in the second plane and/or a third laser to provide the third laser beam in the third plane.

14. The system of claim 13, further comprising a treatment machine comprising a treatment beam source and a patient support unit, and the lasers and the treatment machine are configured and/or arranged such that the target point, of the first, second, and third planes, coincides with the isocenter of the treatment beam source.

15. (canceled)

16. A non-transitory computer readable medium comprising instructions which, when executed by at least one processor, cause the at least one processor to:
acquire a first image by a camera of an AR system with an augmented reality device, wherein the first image depicts a first projection of a first laser beam onto a surface of a subject, the first projection being in the shape of a first line representing the intersection of a first plane with the surface of the subject, the first plane arranged such that it comprises or has a fixed spatial relation with respect to a target point;
determine, based at least on a shape and/or position of the first line in the first image, a first component of a position of the target point in AR device coordinates; and
adjust a registration of a virtual image, which is to be overlaid with the field of view of the AR device, based on the first component of the position of the target point in AR device coordinates.
